(19) United States
(12) Patent Application Publication
(10) Pub. No.: US 2022/0336093 A1
(43) Pub. Date: Oct. 20, 2022

(54) INFORMATION PROCESSING SYSTEM, INFORMATION PROCESSING METHOD, AND STORAGE MEDIUM

(71) Applicant: CANON KABUSHIKI KAISHA, Tokyo (JP)

(72) Inventor: Yukiteru Masuda, Kanagawa (JP)

(21) Appl. No.: 17/720,178

(22) Filed: Apr. 13, 2022

(30) Foreign Application Priority Data
Apr. 16, 2021 (JP) 2021-069722
Jan. 31, 2022 (JP) 2022-013183

(51) Int. Cl. G16H 40/63

(52) U.S. Cl. CPC G16H 40/63

(57) ABSTRACT

An information processing system includes an event information acquisition unit configured to acquire event information about an event that affects a first type of biological information, an abnormality detection unit configured to set a threshold for the first type of biological information based on at least the event information and to compare the piece of the first type of biological information with the threshold to detect whether an abnormality exist in the first type of biological information and a notification unit configured to provide a notification that an abnormality in the first type of biological information has been detected, to the subject and/or a person involved in acquisition of the first type of biological information.

### BACKGROUND

### Field

The present disclosure relates to an information processing system for detecting abnormality in biological information about subjects, an information processing method, and storage medium.

### Description of the Related Art

There is known a technique of promptly detecting abnormalities in biological information about subjects by acquiring the biological information over time and providing a notification in response to the acquired biological information being more than a reference threshold. For example, Japanese Patent Application Laid-Open No. 2020-14732 discusses a technique of detecting an abnormality in current biological information by preliminarily acquiring biological information, calculating a reference threshold, and comparing the current biological information with the reference threshold. In the technique discussed in Japanese Patent Application Laid-Open No. 2020-14732, some types of biological information can greatly vary due to the occurrence of events, such as medication and eating, and such a great variation can be erroneously detected as an abnormality in biological information even in a normal condition in a subject.

### SUMMARY

Various embodiments of the present disclosure are directed to accurate detection of abnormalities in biological information even if certain factors affect the biological information.

According to one embodiment of the present disclosure, an information processing system includes a setting unit configured to make a setting to detect an abnormality in a first type of biological information of a plurality of types of biological information, a biological information acquisition unit configured to acquire a piece of the first type of biological information from a subject, an event information acquisition unit configured to acquire event information about an event that affects the first type of biological information, an abnormality detection unit configured to set a threshold for the first type of biological information based on at least the event information and to compare the piece of the first type of biological information with the threshold to detect whether an abnormality exists in the first type of biological information, and a notification unit configured to provide a notification that an abnormality in the first type of biological information has been detected, to the subject and/or a person involved in acquisition of the first type of biological information.

Further features of the present disclosure will become apparent from the following description of example embodiments with reference to the attached drawings.

### BRIEF DESCRIPTION OF THE DRAWINGS

FIG. 1 is a block diagram illustrating a configuration example of an information processing system according to a first example embodiment.

FIG. 2 is a block diagram illustrating another configuration example of the information processing system according to the first example embodiment.

FIG. 3 is a flowchart illustrating processing in setting a threshold in the information processing system according to the first example embodiment.

FIG. 4 is a flowchart illustrating processing in determining an abnormality in the information processing system according to the first example embodiment.

FIG. 5 is a block diagram illustrating a configuration of a modified example of the information processing system according to the first example embodiment.

FIG. 6 is a flowchart illustrating processing in setting a threshold in an information processing system according to a second example embodiment.

### DESCRIPTION OF THE EMBODIMENTS

Some example embodiments of the present disclosure will be described below with reference to the drawings. In general, like numbers refer to like components, and redundant descriptions thereof will be omitted. The scope of the invention is not limited to the illustrated examples.

An information processing system according to various example embodiment will be described below. According to one example embodiment, one or more pieces of biological information about a subject, as well as information about an active state (such as engaging in an exercise, sleeping, or taking a bath) of the subject, as an event that could affect the biological information, to thereby detect an abnormality in the biological information of the subject.

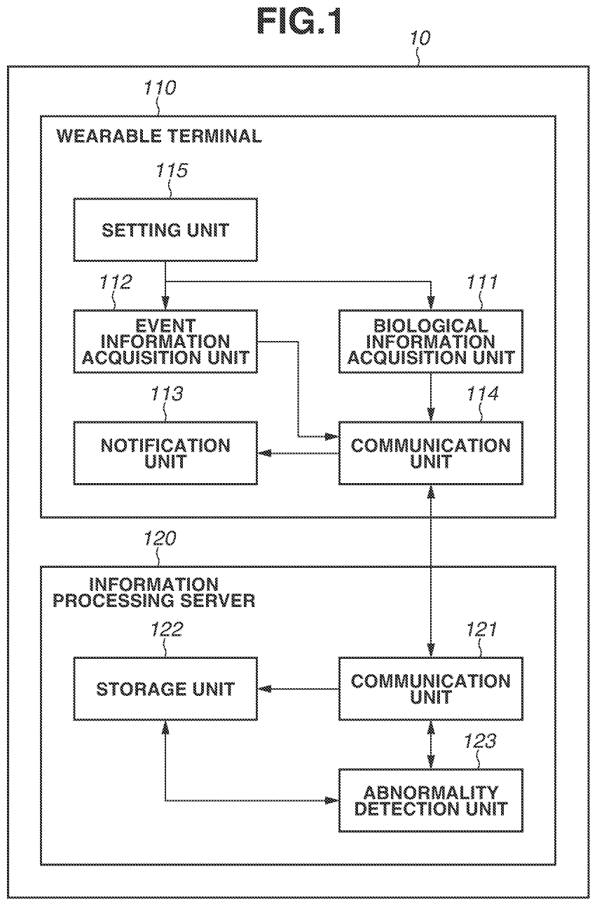

FIG. 1 illustrates a configuration example of an information processing system 10 according to the present example embodiment. The information processing system 10 according to the present example embodiment includes a wearable terminal 110 and an information processing server 120. The wearable terminal 110 and the information processing server 120 are connected through communication units 114 and 121 that are included in the wearable terminal 110 and the information processing server 120, respectively.

The wearable terminal 110 measures one or more types of biological information about the subject that wears the wearable terminal 110 over time. The wearable terminal 110 is, for example, a watch-type terminal. The wearable terminal 110 includes a biological information acquisition unit 111, an event information acquisition unit 112, a notification unit 113, and the communication unit 114. The biological information acquisition unit 111 acquires a piece of biological information about the subject. The event information acquisition unit 112 acquires event information about an event that could affect the biological information about the subject. Upon detection of an abnormality in the biological information about the subject, the notification unit 113 provides a notification to the subject or a person involved in acquisition of the biological information. The communication unit 114 communicates with an external apparatus.

Examples of an involved person include the family of the subject and a primary care doctor for the subject. Although not illustrated, the notification unit 113 can be included in an information terminal such as a mobile terminal owned by the involved person.

The wearable terminal 110 transmits the biological information about the subject acquired by the biological information acquisition unit 111 and the event information acquired by the event information acquisition unit 112 to the information processing server 120 through the communication unit 114. The communication unit 114 receives an abnormality detection signal from the information processing server 120, and transmits the abnormality detection signal to the notification unit 113. Upon reception of the abnormality detection signal, the notification unit 113 provides a notification about abnormality detection information by a predetermined method.

The information processing server 120 acquires the biological information and the event information from the communication unit 114 in the wearable terminal 110 over time through the communication unit 121. The biological information and the event information are transmitted to a storage unit 122 and an abnormality detection unit 123 as time-series data. The storage unit 122 includes a storage area for storing data, and stores the time-series data in the storage area. The abnormality detection unit 123 receives the time-series data (biological information and event information) from the communication unit 121 and the storage unit 122, and sets a threshold for target biological information based on the time-series data received from the storage unit 122. Further, the abnormality detection unit 123 compares the biological information with the threshold based on the biological information and the event information at the time (current time) when the biological information and the event information are received from the communication unit 121. If the biological information exceeds the threshold, the abnormality detection unit 123 transmits the abnormality detection signal to the wearable terminal 110 through the communication unit 121.

Detailed configurations of the wearable terminal 110 and the information processing server 120 according to the present example embodiment will be described.

The biological information acquisition unit 111 includes a sensor for target biological information such as a body temperature, a blood pressure, a blood sugar level, an amount of activity, a heart rate, a cardiac potential, or a blood oxygen saturation level. In other words, the biological information indicates at least one of a body temperature, a blood pressure, a blood sugar level, an amount of activity, a heart rate, a cardiac potential, or a blood oxygen saturation level. The biological information acquisition unit 111 acquires the biological information through the sensor. While the present example embodiment illustrates an example where a heart rate and other types of information are measured, one or more types of sensors may be used.

A timing of biological information acquisition by the biological information acquisition unit 111 will now be described. Since the wearable terminal 110 is worn on the subject's body, the biological information acquisition unit 111 continuously acquires the biological information about the subject while the subject is wearing the wearable terminal 110. Thus, the biological information about the subject is constantly monitored. Further, the biological information acquisition unit 111 may acquire the biological information about the subject at a preliminarily set acquisition timing. For example, if the subject gets up at six o'clock in the morning, the biological information acquisition unit 111 acquires the biological information about the subject between six o'clock and seven o'clock in the morning. If the subject has breakfast at seven o'clock in the morning, the biological information acquisition unit 111 acquires the biological information about the subject between seven o'clock and eight o'clock in the morning. The biological information acquisition unit 111 can select and acquire, at a preliminarily set acquisition timing (time), the type of biological information to be monitored from among a plurality of types of biological information, including a body temperature, a blood pressure, a blood sugar level, an amount of activity, a heart rate, a cardiac potential, and a blood oxygen saturation level.

In addition, the subject can set the biological information acquisition timing before the subject starts a predetermined event. For example, before engaging an exercise, the subject can set the biological information acquisition timing so that the biological information about the subject can be acquired while the subject is making the exercise. Before going to bed, the subject can set the biological information acquisition timing so that the biological information about the subject can be acquired while the subject is sleeping. In this manner, the subject can arbitrarily set the biological information acquisition time or timing for the biological information acquisition unit 111. The subject can also arbitrarily set the time or timing for the biological information acquisition unit 111 to acquire the type of biological information to be monitored from among a plurality of types of biological information.

The information processing system 10 according to the first example embodiment also includes a setting unit 115 that makes a setting to detect an abnormality in the first type of biological information from among a plurality of types of biological information. The biological information acquisition unit 111 acquires one or more pieces of the first type of biological information from among a plurality of types of biological information acquired from the subject. The event information acquisition unit 112 acquires event information about an event that affects the first type of biological information. The abnormality detection unit 123 sets a threshold for the first type of biological information based on the event information and compares the piece of the first type of biological information with the threshold to thereby detect an abnormality in the first type of biological information.

The biological information acquisition unit 111 acquires information about a plurality of types of biological information such as a body temperature, a blood pressure, a blood sugar level, an amount of activity, a heart rate, a cardiac potential, and a blood oxygen saturation level. The setting unit 115 makes a setting for acquiring the first type of biological information (specific biological information) from among a plurality of types of biological information. In other words, the setting unit 115 can select the type of biological information to be monitored by the subject. For example, if the subject wishes to monitor his or her heart rate, the setting unit 115 makes a setting to detect an abnormality in the heart rate, thereby making it possible to detect an abnormality in the set type of biological information, here, the heart rate. Thus, the heart rate of the subject can be monitored. If the subject wishes to monitor his or her blood pressure and blood sugar level, the setting unit 115 makes a setting to detect an abnormality in the blood pressure and blood sugar level, thereby making it possible to detect an abnormality in the blood pressure and blood sugar level.

The event information acquisition unit 112 includes an input interface. The subject inputs event information via the input interface. Examples of the event include medication, eating, sleeping, engaging an exercise, and taking a bath. In other words, the event information indicates at least one of medication, eating, sleeping, engaging in an exercise, or taking a bath. Before a predetermined event starts, the subject inputs event information via the input interface. For example, before taking medication, the subject inputs event information about the medication via the input interface. Before eating, the subject inputs event information about the diet via the input interface.

The event information acquisition unit 112 can acquire event information at a preliminarily set acquisition timing (time). In this case, the preliminarily set time is associated with the event information. For example, if the subject has breakfast at seven o'clock in the morning, the event information acquisition unit 112 periodically acquires event information about having breakfast at seven o'clock in the morning. If the subject takes a bath at eight o'clock in the evening, the event information acquisition unit 112 periodically acquires event information about taking a bath at eight o'clock in the evening.

If the subject will not take a bath at eight o'clock in the evening, the subject can change the time when event information about taking a bath is acquired by the event information acquisition unit 112. For example, if the subject is to take a bath at nine o'clock in the evening, the subject inputs the time (nine o'clock in the evening) to take a bath via the input interface. The event information acquisition unit 112 acquires event information about taking a bath at nine o'clock in the evening.

Further, the event information acquisition unit 112 may include a sensor for detecting an exercise event about the subject. For example, the event information acquisition unit 112 includes an acceleration sensor for detecting an exercise event about the subject. The acceleration sensor measures the acceleration of the subject, which makes it possible to measure the exercise made by the subject. Thus, the use of the acceleration sensor allows the event information acquisition unit 112 to detect an exercise event such as walking or workout. The sensor for detecting an event about the subject is not limited to an inertia sensor such as an acceleration sensor, but instead may be a position sensor such as a global positioning system (GPS), a sensor that uses a subject image captured by a monitoring camera, or other sensors.

The notification unit 113 has a notification function for providing the subject with a notification that an abnormality has occurred. Upon reception of the abnormality detection signal described above, the notification unit 113 provides the subject with a notification that an abnormality has occurred.

Examples of the notification function of the notification unit 113 include a sound alert function, a screen display function for displaying the occurrence of an abnormality on an interface, and a vibration function. As illustrated in FIG. 1, the wearable terminal 110 includes the notification unit 113 worn on the subject's body. This configuration allows the subject to recognize the abnormality detection information.

The communication unit 114 is connected to a network. Examples of the network include a communication network, such as a wireless communication (Wi-Fi®) network, the Internet, a wireless base station, a provider, and a communication line. The communication unit 114 transmits the biological information and the event information to the information processing server 120. The communication unit 114 may transmit the biological information and the event information as time-series data.

If a biological signal value that exceeds the threshold set by the abnormality detection unit 123 to be described below is detected, the communication unit 114 receives the abnormality detection signal from the information processing server 120.

Like the communication unit 114, the communication unit 121 is connected to the network. The communication unit 121 and the communication unit 114 communicates with each other via the network. The communication unit 121 receives the biological information and the event information transmitted from the wearable terminal 110.

If the biological signal value that exceeds the threshold set by the abnormality detection unit 123 to be described below is detected, the abnormality detection signal is transmitted to the communication unit 114.

The storage unit 122 includes a storage area for storing data. The storage unit 122 is, for example, a magnetic disk (hard disk drive (HDD)) that can store large-volume data. The storage unit 122 stores the biological information and the event information received from the communication unit 121 in the storage area. If the biological information and the event information are time-series data, the storage unit 122 stores the biological information and the event information as time-series data.

The abnormality detection unit 123 receives the biological information and the event information from the communication unit 121 and the storage unit 122. The abnormality detection unit 123 sets a threshold for abnormal values in biological information using the biological information and the event information.

For example, the abnormality detection unit 123 obtains the average and variance of biological information in a predetermined period when no event occurs from the time-series data of biological information and event information on a previous day (the day before the relevant day) stored in the storage unit 122. The abnormality detection unit 123 may obtain the average and variance of biological information in the predetermined period when no event occurs from the time-series data of biological information and event information during a previous period (day of week, month, year) stored in the storage unit 122.

The abnormality detection unit 123 sets a threshold based on the values of the average and variance of biological information. Specifically, the abnormality detection unit 123 sets as a threshold for biological information to be measured, for example, a value less than 5% as a significance level in the chi-square distribution with a degree of freedom of 1 for the negative log likelihood of biological information values measured with respect to the average and variance of biological information when no event occurs, based on Hotelling's theory.

If an event that affects biological information occurs, the threshold for the biological information affected by the event may be changed based on the value and the period set for each event. For example, if it is known that the body temperature of the subject is positively affected due to the occurrence of an exercise event of engaging in an exercise, the threshold for the body temperature may be increased upon occurrence of an exercise event. If it is known that the blood pressure of the subject is positively affected by an exercise event of engaging in an exercise, the threshold for the blood pressure may be increased upon occurrence of an exercise event. If it is known that the blood sugar level of the subject is positively affected by an eating event of taking a meal, the threshold for the blood sugar level may be increased upon occurrence of an eating event.

The processing of changing the threshold for a type of biological information based on event information as described above makes it possible to prevent the occurrence of an event of erroneously detecting as an abnormality, for example, a temporary increase in the body temperature, or an increase in the blood pressure or blood sugar level due to exercising.

Assuming that a plurality of types of biological information is to be monitored, the abnormality detection unit 123 can set the threshold for each of the types of biological information depending on the type of event information acquired by the event information acquisition unit 112. For example, if an eating event is acquired by the event information acquisition unit 112, the abnormality detection unit 123 changes the threshold for the blood sugar level that is affected by an eating event, and does not change the threshold for the blood pressure that is not affected by an eating event. If an exercise event is acquired by the event information acquisition unit 112, the abnormality detection unit 123 does not change the threshold for the blood sugar level that is not affected by an exercise event, and changes the threshold for the blood pressure that is affected by an exercise event. In other words, the abnormality detection unit 123 changes the threshold for the type of biological information that is affected by the event acquired by the event information acquisition unit 112, and does not change the threshold for second biological information that is not affected by the event.

In this case, the setting unit 115 can select the type of biological information to be monitored by the subject. If the type of biological information to be monitored is selected by the setting unit 115, the abnormality detection unit 123 extracts event information that affects the selected type of biological information and sets a threshold for the event information. For example, if the subject wishes to monitor his or her heart rate, the heart rate is selected by the setting unit 115. Since events of engaging in an exercise and taking a bath affect the heart rate, the abnormality detection unit 123 increases the threshold for the heart rate with the events of engaging in an exercise and taking a bath. In this manner, the setting unit 115 makes an appropriate setting for detecting an abnormality in the heart rate, thereby making it possible to detect an abnormality in the heart rate more effectively. If the setting unit 115 sets the type of biological information to be monitored using the heart rate as a monitoring target, the setting unit 115 can make a setting for excluding the second type of biological information (blood pressure, pulse, etc.) different from the first type of biological information from the monitoring target. The event information acquisition unit 112 can also make a setting for preventing event information (exercise) that does not affect the first type of biological information from being acquired.

If the subject wishes to monitor his or her blood sugar level, the setting unit 115 selects the blood sugar level. Since an eating event affects the blood sugar level, the abnormality detection unit 123 increases the threshold for the blood sugar level with eating events.

If the setting unit 115 makes a setting for detecting an abnormality in the blood sugar level as described above, an abnormality in the blood sugar level can be detected. If the setting unit 115 sets the first type of biological information using the blood sugar level as a monitoring target, the setting unit 115 can make a setting for excluding the biological information (blood pressure, pulse, etc.) of another type different from the first type of biological information from the monitoring target.

That means that the abnormality detection unit 123 sets the threshold for an abnormal value in the first type of biological information using the first type of biological information set as a monitoring target and event information that affects the first type of biological information. On the other hand, the abnormality detection unit 123 does not set the threshold for an abnormal value based on event information that affects biological information different from the first type of biological information, where the first type of biological information has been set as the monitoring target.

If a value of one or more pieces of the first type of biological information exceeds the threshold, the abnormality detection unit 123 transmits an abnormality detection signal to the notification unit 113 through the communication unit 121. If a value of a plurality of pieces of the first type of biological information exceeds the threshold, the abnormality detection unit 123 transmits an abnormality detection signal together with an identification of the first type as being the type of the pieces of biological information. For example, if the subject monitors his or her blood pressure and blood sugar level and a value of a blood pressure reading exceeds the threshold, the abnormality detection unit 123 transmits an abnormality detection signal together with the type information (blood pressure) about the biological information. If a value of the blood sugar level exceeds the threshold, the abnormality detection unit 123 transmits an abnormality detection signal together with the type information (blood sugar level) of the biological information.

According to one embodiment, arithmetic circuits used in the biological information acquisition unit 111, the event information acquisition unit 112, the notification unit 113, the communication unit 114, the communication unit 121, the storage unit 122, and the abnormality detection unit 123 may take the form of processors designed for exclusive use. Components of the arithmetic circuits may be composed of different hardware modules. At least some of the components of the arithmetic circuits may be composed of a single hardware module. While the present example embodiment illustrates various configuration examples including the wearable terminal 110 and the information processing server 120, the present invention is not limited to this configuration. For example, in other embodiments, an apparatus may include all components in the wearable terminal 110, or the components included in the wearable terminal 110 may be included in the information processing server 120.

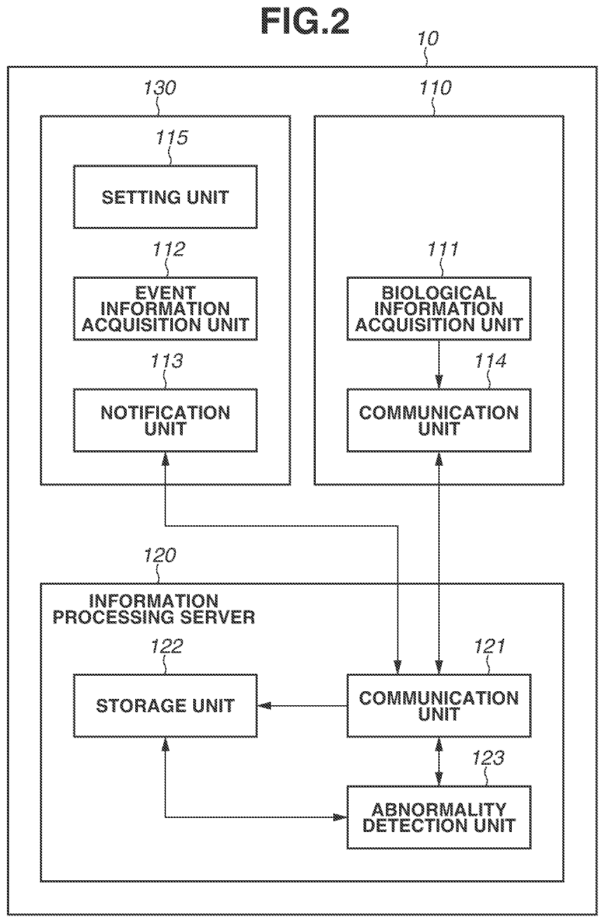

Moreover, alternatively, the event information acquisition unit 112 and the notification unit 113 may be installed in an information terminal. FIG. 2 illustrates a configuration in which an information terminal 130 includes the event information acquisition unit 112 and the notification unit 113. The information terminal 130 could be, for example, a personal computer (PC) terminal, a mobile terminal such as a smartphone, a notebook terminal, or a tablet terminal.

The wearable terminal 110 includes the biological information acquisition unit 111 that acquires biological information about the subject, and the communication unit 114 that communicates with an external apparatus.

The information terminal 130 includes the event information acquisition unit 112 that acquires event information about an occurrence timing of an event that affects biological information about the subject, and the notification unit 113 that provides a notification to the subject or a person involved in acquisition of the biological information when an abnormality in the biological information about the subject is detected. The information terminal 130 has a function of communicating with an external apparatus.

The event information acquisition unit 112 and the notification unit 113 have the same functions as those of the event information acquisition unit 112 and the notification unit 113 illustrated in FIG. 1, and thus the descriptions thereof will be omitted.

If a value of a piece of biological information exceeds the threshold, the abnormality detection unit 123 in the information processing server 120 transmits an abnormality detection signal to the notification unit 113 in the information terminal 130 through the communication unit 121. The notification unit 113 in the information terminal 130 provides the subject with a notification that an abnormality has occurred.

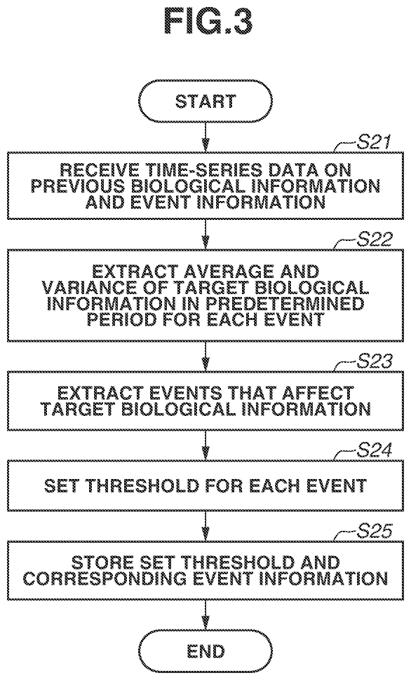

Next, processing for the information processing server 120 to determine a threshold for biological information according to the present example embodiment will be described with reference to a flowchart illustrated in FIG. 3. The information processing server 120 receives an instruction from the subject and starts the processing in the flowchart illustrated in FIG. 3.

In step S21, the abnormality detection unit 123 receives time-series data on previous biological information (e.g., heart rate) and event information stored in the storage unit 122.

In step S22, the abnormality detection unit 123 extracts the average and variance of biological information in each event from the time-series data received in step S21. In this case, the average and variance of target biological information in a predetermined period are extracted for each event.

In step S23, the abnormality detection unit 123 compares the average and variance of biological information extracted in step S22 with the average and variance of biological information in a state where no event is detected, thereby extracting events with a significant difference as events that affect the biological information.

In step S24, the abnormality detection unit 123 sets a threshold for the biological information for each event that affects the biological information, based on the average and variance calculated in step S22.

In step S25, the abnormality detection unit 123 transmits the threshold set in step S24 and the corresponding event information to the storage unit 122, and the storage unit 122 stores the received information in the storage area.

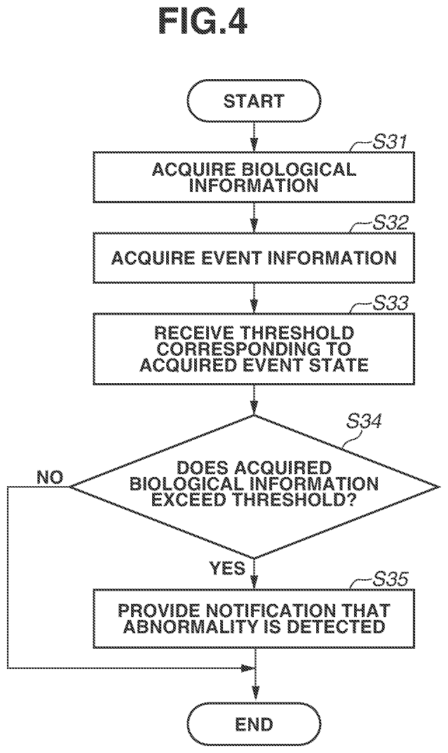

Next, processing for the information processing system 10 to detect an abnormality in biological information and provide a notification according to the present example embodiment will be described with reference to a flowchart illustrated in FIG. 4. The wearable terminal 110 and the information processing server 120 receive an instruction from the subject and start the processing in the flowchart illustrated in FIG. 4.

In step S31, the biological information acquisition unit 111 starts acquisition of biological information of a first type (e.g., heart rate) about the subject, and transmits the acquired biological information to the information processing server 120 through the communication unit 114.

In step S32, the event information acquisition unit 112 acquires current event information (medication, eating, sleeping, engaging in an exercise, taking a bath, etc.) input by the subject via the input interface. If no information is input, it is assumed that no event has occurred. The event information acquisition unit 112 transmits the acquired event information to the information processing server 120 through the communication unit 114.

In step S33, the abnormality detection unit 123 receives the threshold for biological information corresponding to the current event state from the storage unit 122 based on the biological information acquired in step S31 and the event information acquired in step S32.

In step S34, the abnormality detection unit 123 compares the biological information acquired in step S31 with the threshold received in step S33. If the biological information falls less than or equal to the threshold (NO in step S34), the biological information and the event information are transmitted to the storage unit 122. The storage unit 122 stores the biological information and the event information, and then the processing is terminated. On the other hand, if the biological information exceeds the threshold (YES in step S34), the abnormality detection unit 123 transmits an abnormality detection signal to the notification unit 113, and then the processing proceeds to step S35.

In step S35, the notification unit 113 after receiving the abnormality detection signal provides a notification that an abnormality has occurred to the subject or a person involved in acquisition of the biological information by sounding an alert or displaying the notification on the interface.

As described above, the information processing system 10 according to the present example embodiment includes the biological information acquisition unit 111 to acquire biological information about a subject, the event information acquisition unit 112 to acquire event information about an event that affects the biological information about the subject, the abnormality detection unit 123 to detect an abnormality in the biological information based on the biological information and the event information, and the notification unit 113 to provide a notification about abnormality detection information to the subject or a person involved in acquisition of the biological information.

Specifically, the information processing system 10 according to the present example embodiment includes the biological information acquisition unit 111 to acquire biological information of a first type from a subject, the event information acquisition unit 112 to acquire event information about an event that affects the first type of biological information, the abnormality detection unit 123 to set a threshold for the first type of biological information based on the event information and compare one or more pieces of the first type of biological information with the threshold to thereby detect an abnormality in the first type of biological information, and the notification unit 113 to provide a notification about abnormality detection information to the subject or a person involved in acquisition of the first type of biological information. Consequently, the information processing system 10 accurately detects an abnormality in biological information even when an event that affects the biological information occurs during acquisition of the biological information.

### Modified Example 1 of the Above Example Embodiment

While the present example embodiment illustrates an example where a heart rate and other types of information are used as biological information to be measured, the present example embodiment is not limited to this example. In addition to or instead of a heart rate, in other embodiments, other types of biological information such as a body temperature, a blood pressure, a blood sugar level, an amount of activity, a cardiac potential, and a blood oxygen saturation level, or any combination of these types of biological information may be used, for example.

While the present example embodiment described above illustrates an example of an event information acquisition method where event information is input from a subject via the input interface, the event information acquisition method is not limited to this example. For example, an event state may be estimated based on other pieces of biological information acquired by the biological information acquisition unit 111 and time information. More specifically, the amount of activity and the body temperature of the subject may be acquired at the same time as biological information about the subject, and if the amount of activity, the heart rate, and the body temperature of the subject are increased at the same time in the daytime, it can be estimated that an exercise event has occurred. If the amount of activity is kept small for a predetermined period at night, for example, it can be estimated that a sleeping event has occurred. Such modifications reduce the frequency at which the subject inputs information about the occurrence of an event, which leads to a reduction in workload on the subject.

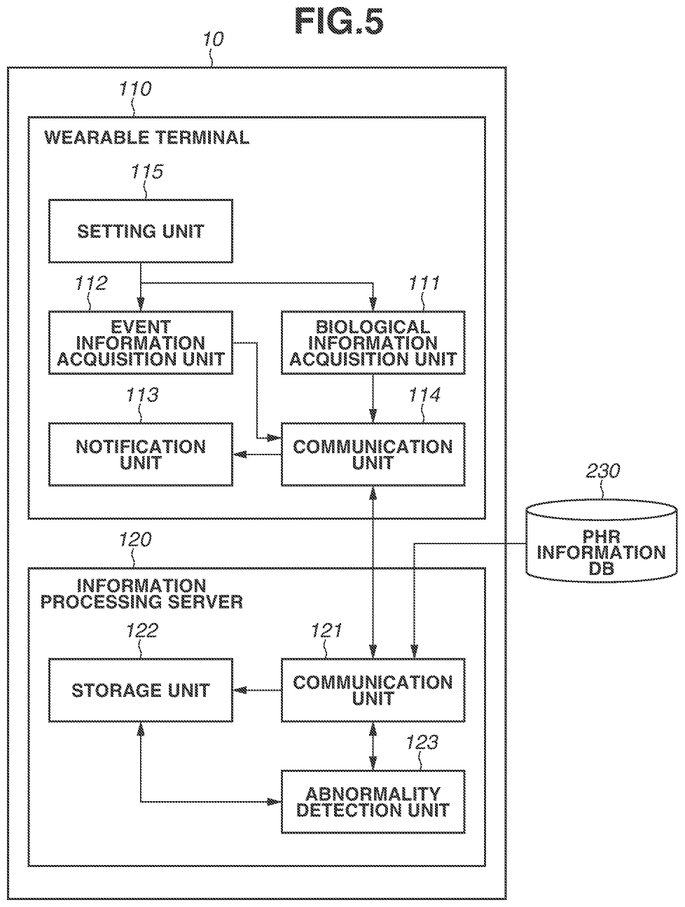

In the present example embodiment, a threshold is determined based on the previous biological information and event information stored in the storage unit 122. However, the threshold determination method is not limited to this method. For example, in other embodiments, as illustrated in FIG. 5, the information acquisition unit (communication unit 121) acquires personal health record (PHR) information (medical information) such as health examination information or medication information about the subject from a PHR information database (DB) 230, and the acquired information is stored in the storage unit 122. For example, in setting the threshold when no event occurs, a value of biological information obtained during a health examination may be loaded from the storage unit 122 and a threshold may be determined based on the value. Such modifications allow setting of a threshold more suited to the actual state in, for example, biological information that needs to be noted in a health examination.

The abnormality detection unit 123 can also set a threshold for abnormal values in the first type of biological information using medication information (for example, type of medication) that affects the first type of biological information. If the event information acquisition unit 112 acquires a medication event that affects the first type of biological information, the abnormality detection unit 123 decreases the threshold for abnormal values in the first type of biological information.

For example, if the blood pressure of the subject is to be monitored as a monitoring target, the abnormality detection unit 123 can also set a threshold for abnormal values in the blood pressure with medication information about a blood pressure medicine that affects the blood pressure. If the event information acquisition unit 112 acquires an event related to administration of a blood pressure medicine that affects the blood pressure, the abnormality detection unit 123 decreases the threshold for the abnormal value in the blood pressure.

Examples of the blood pressure medicine include Ca antagonist, angiotensin receptor blocker (ARB), angiotensin-converting enzyme (ACE) inhibitor, β-blocker, and diuretic. Since various types of blood pressure medicines have different efficacies, the abnormality detection unit 123 can also set a threshold for abnormal values in the blood pressure depending on the type of the blood pressure medicine. In other words, the abnormality detection unit 123 can set a threshold for abnormal values in the first type of biological information depending on the type of the medication information.

Further, the subject periodically (e.g., every day) acquire a blood pressure medicine that affects blood pressure. If no medication event is acquired from the event information acquisition unit 112, the notification unit 113 can provide the subject with a notification to urge the subject to take the blood pressure medicine. In other words, if no medication event is acquired from the event information acquisition unit 112, the notification unit 113 can provide the subject with a notification to urge the subject to take a medicine. In this case, the notification unit 113 provides the subject with a notification by a notification method different from the above-described abnormality occurrence notification method. Thus, this configuration allows the subject to be urged to take a medicine.

In the present example embodiment, the information acquisition unit (communication unit 121) is provided that acquires medical information about the subject from the PHR information DB 230. The abnormality detection unit 123 detects an abnormality in biological information based on the biological information, event information, and medical information. The abnormality detection unit 123 sets a threshold for detecting an abnormality in the biological information based on the event information and the medical information. The abnormality detection unit 123 compares the biological information with the threshold, thereby detecting an abnormality in the biological information.

While the present example embodiment described above illustrates the method of setting a threshold for biological information as an abnormality detection method, the abnormality detection method is not limited to this method. For example, a difference in rising gradient of biological information (values) immediately after an event has occurred may be used, or a machine learning model for determining a normal state or an abnormal state may be used.

While the present example embodiment described above illustrates an example where the wearable terminal 110 is a wristwatch-type terminal, the present example embodiment is not limited to this example. Any terminal may be used, as long as the terminal can measure biological information about the subject over time. For example, a glasses-type terminal, a terminal that is hung from the neck of the subject may be used. More alternatively, a terminal configured to acquire biological information about a subject without wearing the terminal may be used. A terminal such as a thermal camera configured to measure the body temperature of a bedridden subject in a non-contact manner, for example, from a bedside may also be used.

The information processing system 10 according to a second example embodiment will be described. The present example embodiment illustrates an example where the blood sugar level of a subject is acquired as biological information about the subject, and an eating state and a medication state are acquired as events that affect the biological information to thereby detect an abnormality in the biological information. In the following description, like numbers refer to like components, and detailed descriptions thereof will be omitted.

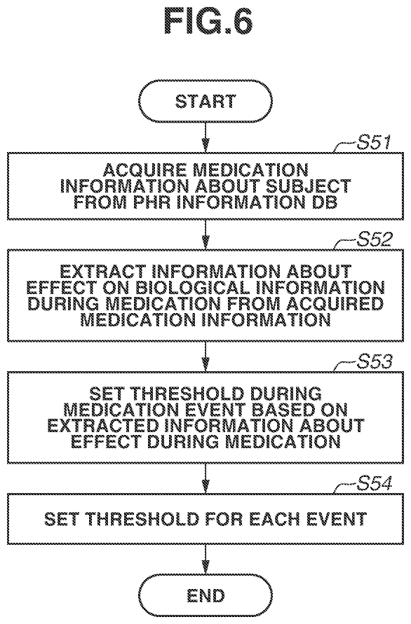

Processing executed in determination by the information processing server 120 of a threshold for the blood sugar level during a medication event according to one example embodiment will be described with reference to a flowchart illustrated in FIG. 6. The information processing server 120 receives an instruction from the subject and executes the processing in the flowchart illustrated in FIG. 6.

In step S51, the abnormality detection unit 123 acquires medication information (drug information), which indicates drugs taken by the subject and affects the blood sugar level, from the PHR information DB 230 through the communication unit 121.

In step S52, the abnormality detection unit 123 extracts information related to the effect on the blood sugar level, such as a medication timing, an amount of effect on the blood sugar level, or an efficacy time duration, from the medication information.

In step S53, the abnormality detection unit 123 sets a threshold for the blood sugar level during the medication event in consideration of the effect on the blood sugar level extracted in step S52, and transmits the threshold to the storage unit 122. In this case, a value that changes over time depending on the lapse of time from medication may be set as the threshold based on a period corresponding to the half-life of drug efficacy.

In step S54, the storage unit 122 stores the information received from the abnormality detection unit 123 in the storage area.

Processing executed in detection by the information processing system 10 of an abnormality in the measured blood sugar level and providing a notification according to the present example embodiment will be described below with reference to the flowchart illustrated in FIG. 4.

The present example embodiment is similar to the first example embodiment described above except that the flowchart for abnormality determination uses the blood sugar level as biological information, eating and medication information as event information, and a threshold based on variations in the blood sugar level during medication as a determination threshold.

In step S31, the biological information acquisition unit 111 acquires the blood sugar level of the subject, and transmits the acquired data to the information processing server 120.

In step S32, the subject inputs eating event and medication event information to the event information acquisition unit 112 via the input interface.

In step S33, the abnormality detection unit 123 receives the threshold for the blood sugar level corresponding to the eating and medication events from the storage unit 122.

In step S34, the abnormality detection unit 123 compares the blood sugar level acquired from the biological information acquisition unit 111 with the threshold. If the blood sugar level falls less than or equal to the threshold (NO in step S34), the blood sugar level and the event information are stored in the storage unit 122, and then the processing is terminated. On the other hand, if the blood sugar level acquired from the biological information acquisition unit 111 exceeds the threshold (YES in step S34), the abnormality detection unit 123 transmits an abnormality detection signal to the notification unit 113, and then the processing proceeds to step S35.

In step S35, the notification unit 113 provides an abnormality detection notification to the subject or a person involved in acquisition of the biological information.

As described above, the information processing system 10 according to the present example embodiment accurately detects an abnormality in biological information even when events that affect target biological information over time, such as eating and medication that affect the blood sugar level, have occurred.

Various embodiments of the present disclosure can also be implemented by processing in which a program for implementing one or more functions according to the example embodiments described above is supplied to a system or an apparatus via a network or a storage medium, and then in which one or more processors in a computer of the system or the apparatus read out and run the program. Various embodiments of the present disclosure can also be implemented by a circuit (e.g., and application specific integrated circuit (ASIC)) for implementing one or more functions according to the example embodiments. This program and a computer-readable storage medium storing the program are also included in various embodiments of the present disclosure.

The above-described example embodiments are merely illustrative of embodiments for carrying out the present invention, and the technical scope of the present invention should not be interpreted as being limited to the example embodiments. That is, various embodiments of the present disclosure can be carried out in various forms without departing from the technical idea or main features of the present invention.

### Other Embodiments

Various embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While example embodiments have been described, it is to be understood that the invention is not limited to the disclosed example embodiments. The scope of the following claims is to be accorded the broadest interpretation so as to encompass all such modifications and equivalent structures and functions.

This application claims the benefit of Japanese Patent Application No. 2021-069722, filed Apr. 16, 2021, and Japanese Patent Application No. 2022-013183, filed on Jan. 31, 2022, which are hereby incorporated by reference herein in their entireties.

What is claimed is:

1. An information processing system comprising:
a setting unit configured to make a setting to detect an abnormality in a first type of biological information of a plurality of types of biological information;
a biological information acquisition unit configured to acquire a piece of the first type of biological information from a subject;
an event information acquisition unit configured to acquire event information about an event that affects the first type of biological information;
an abnormality detection unit configured to set a threshold for the first type of biological information based on at least the event information and to compare the piece of the first type of biological information with the threshold to detect whether an abnormality exists in the first type of biological information; and
a notification unit configured to provide a notification that an abnormality in the first type of biological information has been detected, to the subject and/or a person involved in acquisition of the first type of biological information.

2. The information processing system according to claim 1, further comprising:
a wearable terminal including the biological information acquisition unit; and
an information processing server including the abnormality detection unit,
wherein the wearable terminal and the information processing server are connected through communication units included in the wearable terminal and the information processing server, respectively.

3. The information processing system according to claim 2, wherein the abnormality detection unit sets the threshold based on the first type of biological information and the event information, compares the piece of the first type of biological information with the threshold, and transmits an abnormality detection signal to the wearable terminal in a case where the piece of biological information exceeds the threshold.

4. The information processing system according to claim 1, wherein the first type of biological information indicates at least one of a body temperature, a blood pressure, a blood sugar level, an amount of activity, a heart rate, a cardiac potential, or a blood oxygen saturation level.

5. The information processing system according to claim 1, wherein the event is at least one of medication, eating, sleeping, engaging in an exercise, or taking a bath.

6. The information processing system according to claim 1, wherein the biological information acquisition unit acquires the piece of the first type of biological information about the subject at a preliminarily set acquisition timing.

7. The information processing system according to claim 1, wherein the event information acquisition unit acquires the event information at a preliminarily set acquisition timing.

8. The information processing system according to claim 1, wherein the event information acquisition unit includes a sensor configured to detect an event relating to the subject.

9. The information processing system according to claim 1, wherein the event information acquisition unit does not acquire event information that has no effect on the first type of biological information.

10. The information processing system according to claim 1, further comprising an information acquisition unit configured to acquire medical information about the subject,
wherein the abnormality detection unit detects the abnormality in the first type of biological information based on the event information and the medical information.

11. The information processing system according to claim 10, wherein the abnormality detection unit sets a threshold for detecting an abnormality in the first type of biological information based on the event information and the medical information, and compares the piece of the first type of biological information with the threshold to detect the abnormality in the first type of biological information.

12. The information processing system according to claim 1, wherein the abnormality detection unit sets a threshold for each of the plurality of types of biological information depending on a type of the event information.

13. The information processing system according to claim 1, wherein the abnormality detection unit changes the threshold for the first type of biological information that is affected by conditions relating to the event information acquired by the event information acquisition unit, and does not change the threshold for a second type of biological information that is not affected by the conditions relating to the event information.

14. The information processing system according to claim 1, wherein the abnormality detection unit sets the threshold based on an average and a variance of the first type of biological information.

15. The information processing system according to claim 3, wherein in a case where an event that affects the first type of biological information occurs, the abnormality detection unit changes the threshold based on a value and a period set for each event.

16. The information processing system according to claim 1, wherein the abnormality detection unit sets a threshold for an abnormal value in the first type of biological information using medication information that affects the first type of biological information.

17. The information processing system according to claim 16, wherein the abnormality detection unit sets the threshold for the abnormal value in the first type of biological information depending on a type of medication information that affects the first type of biological information.

18. The information processing system according to claim 1, wherein in a case where no medication event is acquired from the event information acquisition unit, the notification unit provides a notification to urge the subject to take a medicine.

19. An information processing method comprising:
making a setting to detect an abnormality in a first type of biological information of a plurality of types of biological information;
acquiring a piece of the first type of biological information from a subject;
acquiring event information about an event that affects the first type of biological information;
setting a threshold for the first type of biological information based on at least the event information and to compare the piece of the first type of biological information with the threshold to detect whether an abnormality exists in the first type of biological information; and
providing a notification that an abnormality in the first type of biological information has been detected, to the subject and/or a person involved in acquisition of the first type of biological information.

20. A non-transitory computer-readable storage medium that stores a program for causing a computer to execute an information processing method according to claim 19.
